(19) United States
(12) Patent Application Publication
(10) Pub. No.: US 2022/0125718 A1
(43) Pub. Date: Apr. 28, 2022

(54) STABILIZED Fc FUSION PROTEIN SOLUTIONS

(71) Applicant: ARECOR LIMITED, Saffron Walden (GB)

(72) Inventors: Jan JEZEK, Saffron Walden (GB); David GERRING, Saffron Walden (GB); Sarah HOWELL, Saffron Walden (GB)

(21) Appl. No.: 17/428,341

(22) PCT Filed: Feb. 5, 2020
(86) PCT No.: PCT/GB2020/050255

§ 371 Date: Aug. 4, 2021

(30) Foreign Application Priority Data
Feb. 5, 2019 (GB) 1901547.8

(51) Int. Cl. A61K 9/08; A61P 19/02; A61K 38/17; A61K 9/00; A61K 47/02

(52) U.S. Cl. CPC A61K 9/08; A61P 19/02; A61K 47/02; A61K 38/1793; A61K 9/0019; A61K 38/1774

(57) ABSTRACT

There is provided, inter alia, an aqueous solution formulation comprising: (i) an Fc fusion protein; and (ii) sulfate ions at a concentration of 5-200 mM to stabilise the Fc fusion protein; which formulation is free of amino acids selected from arginine, lysine and proline, and salts thereof, and which formulation is free of magnesium ions.

### BACKGROUND OF THE INVENTION

When formulated as aqueous solutions, Fc fusion proteins are susceptible to structural degradation during storage. The processes involved in protein degradation can be divided into physical (e.g. loss of quaternary, tertiary or secondary structure, aggregation, particle formation) and chemical (i.e. processes involving a covalent change such as deamidation, aspartate isomerization, oxidation, hydrolytic clipping etc.). Each of the degradants (e.g. soluble aggregated species, insoluble aggregated species and chemically modified variants) can impact the biological activity, toxicity or immunogenicity of the Fc fusion protein.

Therefore, the level of all degradants has to be kept within the tight specifications that are set for each Fc fusion protein product. The rates of the degradation processes depend on temperature and Fc fusion proteins are generally more stable at lower temperatures. Consequently, commercial Fc fusion protein products must typically be stored refrigerated. However, with increasing trend toward subcutaneous products that can be self-administered by the patient, there is a strong need to develop Fc fusion protein products that can be used outside the cold chain, at least for a period of time, such as 2 weeks, such as 4 weeks, such as 12 weeks or more. The ability to store the product outside the cold chain often results in considerable improvement in convenience for the patient during the in-use period. Allowed excursions outside the cold chain can also significantly improve shipment logistics.

The present invention addresses the problem of instability of Fc fusion proteins in particular the problem of Fc fusion protein degradation and especially aggregation.

WO2003/072060 (Immunex) discloses formulations of proteins comprising an Fc domain and which comprise L-arginine as an aggregation inhibitor.

WO2012/143418 (Sandoz) discloses formulations of etanercept which comprise an amino acid selected from lysine and proline as an aid to improving stability.

WO2013/006454 (Biogen) discloses formulations of etanercept which contain less than 1 mM of L-arginine and which comprise salt in an amount sufficient to prevent aggregation of the etanercept.

### SUMMARY OF THE INVENTION

The present invention addresses the problem of instability of Fc fusion proteins. In one embodiment, the invention relates to an aqueous solution formulation comprising (i) an Fc fusion protein; and

(ii) sulfate ions at a concentration of 5-200 mM to stabilise the Fc fusion protein;
which formulation is free of amino acids selected from arginine, lysine and proline, and salts thereof, and which formulation is free of magnesium ions.

### DETAILED DESCRIPTION OF THE INVENTION

The present invention relates to the discovery that an aqueous solution of an Fc fusion protein can be stabilized by sulfate ions at a concentration of 5-200 mM.

The term “aqueous solution formulation”, as used herein, refers to a solution in water, preferably distilled water, deionized water, water for injection, sterile water for injection or bacteriostatic water for injection. The aqueous solution formulation of the invention include dissolved Fc fusion protein, sulfate ions, optionally, one or more additives and/or excipients, and are free of amino acids selected from arginine, lysine and proline, and salts thereof, and are free of magnesium ions. The aqueous solution formulations can also include one or more components, such as additives or excipients, which are partially dissolved or undissolved. The presence of such component or components will result in a multi-phase formulation, such as a suspension or an emulsion. Preferably, the aqueous solution formulation of the invention is a homogeneous solution, as determined by eye or by light-scattering.

The term “Fc fusion protein”, as used herein refers to protein comprising a heterologous polypeptide genetically linked to the Fc portion of an immunoglobulin, or a derivative thereof. By “heterologous polypeptide” is meant a polypeptide not naturally found genetically linked with the Fc portion and is typically not a polypeptide derived from an antibody. By “genetically linked” in respect of two proteins/polypeptides is meant that the two proteins/polypeptides are linked by a peptide bond and can be encoded by a single nucleic acid molecule. For example, the heterologous polypeptide is capable of binding a ligand, preferably a specific ligand. The heterologous polypeptide may be capable of interacting with another protein, for example, a protein that has a role in the human body (such as, without limitation, a cytokine). More generally, the heterologous polypeptide may be therapeutically useful. Exemplary such heterologous polypeptides include, without limitation, a cytokine, a blood clotting factor or a growth factor or a functional fragment or domain thereof. A further example is a GLP-1 agonist. Examples of derivatives include conjugated derivatives e.g. such as an Fc fusion protein conjugated to another moiety. Such moieties include chemically inert polymers such as PEG.

In certain embodiments, two Fc fusion proteins can associate e.g. via disulfide bonds to form a dimeric protein.

In certain embodiments the Fc region of an immunoglobulin is the Fc region of a IgG, including IgG1, IgG2, IgG3 or IgG4, IgM, IgA, such as IgA1 or IgA2, IgD, IgE or IgY. Most suitably the Fc region of an immunoglobulin is the Fc region of an IgG, especially IgG1,

In some embodiments, the Fc fusion protein contains one or more modifications within the Fc region that alters one or more properties of the Fc fusion protein, such as serum half-life, complement fixation, Fc receptor binding, and/or effector function (e.g. antigen-dependent cellular cytotoxicity).

In an embodiment, the Fc fusion protein comprises a heterologous peptide which is capable of binding a ligand i.e. is ligand binding.

In an embodiment, the heterologous polypeptide is capable of binding to tumour necrosis factor (TNF) e.g. TNFα, and for example may comprise a TNF receptor, e.g. TNF receptor 2, especially a soluble form thereof.

In an embodiment, the heterologous polypeptide is capable of binding to CD80 or CD86 and, for example, comprises the extracellular domain of CLTA-4 or a portion thereof.

In an embodiment, the heterologous polypeptide is capable of binding to VEGF and for example comprises the extracellular domain of VEGFR1 and/or VEGR2 or a portion thereof.

In an embodiment, the heterologous polypeptide is capable of binding to IL-1 and for example comprises the extracellular domain of IL-1R11 and/or IL-1RAcP or a portion thereof.

In an embodiment, the heterologous polypeptide is capable of binding to a thrombopoetin receptor such as c-Mpl.

In an embodiment, the heterologous polypeptide is a blood clotting factor such as Factor VIII or Factor IX or a portion thereof.

In an embodiment, the heterologous polypeptide is a hActRIIb protein or a derivative thereof.

In an embodiment, the heterologous polypeptide is a GLP-1 agonist such as dulaglutide.

Example Fc fusion proteins include etanercept, abatacept and belatacept. Etanercept is particularly of interest.

Further examples include aflibercept, rilonacept, romiplostim, eloctate, luspatercept and alprolix.

In certain embodiments, the Fc fusion protein is substantially pure, that is, the formulation comprises a single Fc fusion protein and no substantial amount of any additional protein. In preferred embodiments, the Fc fusion protein comprises at least 99%, preferably at least 99.5% and more preferably at least about 99.9% of the total protein content of the formulation. In preferred embodiments the Fc fusion protein is sufficiently pure for use as in a pharmaceutical formulation.

The Fc fusion protein is preferably a therapeutic Fc fusion protein. Such an Fc fusion protein has a desirable therapeutic or prophylactic activity and is indicated for the treatment, inhibition or prevention of a disease or medical disorder.

The Fc fusion protein is suitably present at a concentration of about 1-400 mg/ml, suitably 20-100 mg/ml, more suitably about 50 mg/ml.

As noted above, the inventors have discovered that the stability of the Fc fusion protein in aqueous solution formulation is improved in the presence of sulfate ions. Suitably the sulfate ions are present in the formulation at a concentration of 25-150 mM e.g. 25-100 mM or 25-90 mM, more suitably 50-100 mM or 50-90 mM, e.g. 50-75 mM. Another range of interest is 50-125 mM e.g. 75-125 mM.

The source of the sulfate ions is suitably a sulfate salt e.g. selected from sodium sulfate, potassium sulfate and ammonium sulfate. The source of the sulfate ions should not be magnesium sulfate.

The inventors have also discovered that the stability of the Fc fusion protein in an aqueous solution formulation may be further improved by the presence of a multivalent anion (or more than one type of multivalent ion) other than sulfate. Thus, aqueous solution formulations of the invention suitably comprise a multivalent anion (or more than one) other than sulfate as a further stabilizing component.

The multivalent anions (other than sulfate) have a charge of at least minus 2 (which may also be written as “−2” or “minus two”). The multivalent anions may, for example, have a charge of minus 2 (“divalent anions”), minus 3 (“trivalent anions”) or minus 4 (tetravalent anions”). In one embodiment, the multivalent anions have a charge of minus 2 and are divalent anions. In another embodiment, the multivalent anions have a charge of minus 3 and are trivalent anions. In a further embodiment, the multivalent anions are a mixture of anions having charge of minus 2 and anions having charge of minus 3, i.e. are a mixture of divalent and trivalent anions. In another embodiment, the multivalent anions are divalent anions, trivalent anions or a mixture thereof. The multivalent anions are species which do not comprise any group capable of forming a positive charge (e.g. by protonation) in the range of pH 4-7 at 25° C. Thus, the multivalent anions do not contain basic nitrogen centres, i.e. nitrogen centres which are capable of being protonated. In particular, the multivalent anions do not contain a quaternary ammonium group (i.e. a positively charged tetra-substituted nitrogen atom). The multivalent anions do not contain protonatable nitrogen centres with pKₐ between 5-8 or 3-9 at 25° C. The multivalent anions are not amino acids and particularly are not one of the 20 natural amino acids in L or D isomeric form or any mixture thereof (including a racemic mixture). Thus, the multivalent anions are not glutamate, aspartate or a mixture thereof. The multivalent anions are not peptides or proteins (i.e. molecules which comprise two or more amino acid residues). In one embodiment, the multivalent anions do not contain nitrogen atoms. The multivalent anions are not nitrogen-containing chelating agents, and in particular are not ethylenediaminetetraacetic acid (EDTA). Suitably the multivalent anions have a molecular weight of less than 500 Da, for example less than 400 Da, less than 300 Da or less than 200 Da.

For example, the multivalent anion is an organic multivalent anion. An example organic multivalent anion is citrate. Further examples include succinate, malate and maleate, particularly maleate. The multivalent anion may also be an inorganic multivalent anion other than sulfate, such as phosphate. Example counter-ions for the multivalent anion include sodium, potassium and ammonium ions, particularly sodium and potassium ions The counter-ion should not be magnesium. For example, the multivalent anion may be employed as sodium citrate, sodium maleate or sodium phosphate.

In an embodiment, the multivalent anion(s) consist of organic multivalent anions i.e. there are no inorganic multivalent anions present in the formulation. In an embodiment, the formulation is free of phosphate ions.

A mixture of multivalent anions may also be used (for example an organic and an inorganic multivalent anion or two organic multivalent anions). Thus, for example, two or more (e.g. two or three) different multivalent anions other than sulfate may be employed. In a preferred embodiment, citrate and maleate as multivalent anions are employed. In an embodiment, phosphate, citrate and maleate as multivalent anions are employed,

The concentration of the (or each) multivalent anion other than sulfate in the solution formulation may for example be about 1-100 mM, suitably about 1-60 mM or 1-50 mM, more suitably about 5-100 mM or 5-60 or 5-50 mM, more suitably about 10-60 or 10-50 mM or 10-30 mM or 10-20 mM or 30-50 mM.

When more than one multivalent ion (other than sulfate) is employed, suitably the total concentration of multivalent ions is about 1-100 mM, suitably about 1-60 mM or 1-50 mM, more suitably about 5-100 mM or 5-60 or 5-50 mM, more suitably about 10-60 or 10-50 mM or 10-30 mM or 10-20 mM or 30-50 mM.

The aqueous solution formulation of the invention may cover a wide range of osmolarity, including hypotonic, isotonic and hypertonic aqueous solutions. Suitably, the aqueous solution formulation of the invention is substantially isotonic. In one embodiment, the aqueous solution formulation of the invention is isotonic. Suitably, the osmolarity of the aqueous solution formulation is selected to minimize pain according to the route of administration e.g. upon injection. Suitable aqueous solution formulations have an osmolarity in the range of about 200 mOsm/L to about 500 mOsm/L. Suitably, the osmolarity is in the range of about 250 mOsm/L to about 350 mOsm/L. More suitably, the osmolarity is about 300 mOsm/L.

Tonicity of the aqueous solution formulation may be adjusted with a tonicity modifier. Tonicity modifiers may be charged or uncharged.

Suitably the aqueous solution formulation according to the invention is isotonic.

In an embodiment, tonicity is adjusted by the presence in the formulation of an uncharged tonicity modifier. The concentration of the tonicity modifier will be adapted according to the tonicifying effect of the sulfate ions and any multivalent anions present in the formulation. For example, if these provide a suitable tonicifying effect then no further tonicity modifier would be required.

Examples of uncharged tonicity modifiers include sugars, sugar alcohols and other polyols, such as sucrose, trehalose, mannitol, raffinose, lactose, dextrose, sorbitol or lactitol, or glycerol or propylene glycol, or polyethylene glycols such as PEG300 or PEG400. In one embodiment, the uncharged tonicity modifier is selected from sucrose, trehalose, mannitol, sorbitol, PEG300 and PEG400. Such an uncharged tonicity modifier when employed may, for example, be present at a concentration of about 10-1000 mM, such as about 50-500 mM, such as about 50-300 mM, such as about 10-300 mM, such as about 10-100 mM, such as about 10-50 mM.

In an embodiment, the aqueous solution formulation is free of xylitol.

Alternatively, tonicity may be adjusted by the presence in the formulation of a charged tonicity modifier. The charged tonicity modifier is a charged substance other than sulfate or a multivalent anion. Examples of charged tonicity modifiers include salts such as a combination of sodium or potassium ions, with chloride, nitrate or acetate ions, especially sodium chloride. The charged tonicity modifier when employed may, for example, be present in the formulation at a concentration of about 25-500 mM, suitably about 50-250 mM, e.g. about 150 mM; or about 10-250 mM such as about 10-100 mM such as about 10-50 mM.

In an embodiment of the invention, the aqueous solution formulation does not contain a charged tonicity modifier and in particular contains less than 5 mM e.g. less than 1 mM sodium chloride e.g. the aqueous solution formulation does not contain sodium chloride.

In an embodiment of the invention, the aqueous solution formulation does not contain an uncharged tonicity modifier.

Suitably the pH of the aqueous solution formulation is between about pH 4.0 and about pH 7.0. In one embodiment the pH is between about pH 4.0 and about pH 6.0 e.g. between about pH 5.0 and about pH 6.0 e.g. between about pH 5.0 and about pH 5.7 e.g. about pH 5.3. In a more preferred embodiment the pH is between about pH 6.0 and about pH 7.0 e.g. between about pH 6.0 and about pH 6.5 e.g. about pH 6.3

In one embodiment the aqueous solution formulation of the invention further comprises a buffer in order to stabilise the pH of the formulation, and which can also be selected to enhance Fc fusion protein stability. A buffer is typically a substance having at least one ionisable group with a pKa within 2 pH units (such as within 1.5 pH units especially within 1 pH unit) of the pH of the formulation (especially as determined at 25° C.). Suitably the buffer is present at a concentration of about 0.5 mM to about 50 mM, such as about 1 mM to about 20 mM, such as about 1 mM to about 10 mM, e.g. 1 mM to about 5 mM e.g. about 2 mM to about 5 mM. An example buffer is acetate. Certain example buffers are multivalent anions. If the buffer is a multivalent anion (e.g. phosphate or citrate) it is treated as a multivalent anion and not a buffer for the purposes of determining the amounts of the components of the formulation.

In an embodiment, the formulation does not contain any buffer which is not a multivalent anion.

The aqueous solution formulations of the invention may optionally comprise a surfactant. In one embodiment, the surfactant is a non-ionic surfactant such as an alkyl glycoside e.g. dodecyl maltoside; a polysorbate surfactant such as polysorbate 80 or polysorbate 20; an alkyl ether of polyethylene glycol e.g. selected from polyethylene glycol (2) dodecyl ether, polyethylene glycol (2) oleyl ether and polyethylene glycol (2) hexadecyl ether; a block copolymer of polyethylene glycol and polypropylene glycol, such as poloxamer 188, poloxamer 407, poloxamer 171 or poloxamer 185; or an alkylphenyl ether of polyethylene glycol, such as 4-(1,1,3,3-tetramethylbutyl)phenyl-polyethylene glycol. 27. Suitably the surfactant is present at a concentration of about 10 μg/mL to about 2000 μg/mL, such as about 50 μg/mL to about 1000 μg/mL, e.g. about 100 μg/mL to about 500 μg/mL

The aqueous solution formulations of the invention may optionally include a preservative, suitably selected from phenol, m-cresol, chlorocresol, benzyl alcohol, propylparaben, methylparaben, benzalkonium chloride and benzethonium chloride. When present, the preservative is at a concentration of about 0.01 mM to about 100 mM. A preservative selected from phenol, m-cresol, chlorocresol, benzyl alcohol, propylparaben, methylparaben may, for example, be present at a concentration of about 10 mM to about 100 mM, such as about 20 mM to about 80 mM e.g. about 25 mM to about 50 mM. A preservative selected from benzalkonium chloride and benzethonium chloride may, for example, be present at a concentration of about 0.01 mM to about 1 mM such as about 0.05 mM to about 0.5 mM e.g. about 0.05 mM to about 0.2 mM.

In an embodiment, the aqueous solution formulations of the invention are free of all amino acids (i.e. free amino acids which do not comprise any peptide bonds), particularly are free of any of the 20 natural amino acids in their L isomeric forms, or corresponding D isomeric forms or any mixtures of isomers thereof (including racemic mixtures).

The term “high molecular weight species” as used herein, refers to any component of the Fc fusion protein content which has an apparent molecular weight at least about double the molecular weight of the parent active Fc fusion protein. That is, high molecular weight species are multimeric aggregates of the parent Fc fusion protein. The multimeric aggregates may comprise the parent Fc fusion protein molecules with considerably altered conformation or they may be an assembly of the parent protein units in the native or near-native conformation. The determination of high molecular weight species can be done using methods known in the art, including size exclusion chromatography, electrophoresis, analytical ultracentrifugation/sedimentation velocity, light scattering, dynamic light scattering and field flow fractionation.

The term “low molecular weight species” as used herein, refers to any component of the Fc fusion protein content which has an apparent molecular weight less than the molecular weight of the parent active Fc fusion protein. That is, low molecular weight species are fragments of the parent Fc fusion protein. The determination of high molecular weight species can be done using methods known in the art, including size exclusion chromatography, electrophoresis, analytical ultracentrifugation/sedimentation velocity, light scattering, dynamic light scattering and field flow fractionation.

The term “related species” as used herein, refers to any component of the Fc fusion protein content formed by a chemical modification of the parent Fc fusion protein, such as deamidated species or oxidised species. Related species are suitably detected by cation-exchange chromatography, anion-exchange chromatography, reversed-phase chromatography or capillary electrophoresis.

Suitably an aqueous solution formulation of the invention is sufficiently stable such that it remains substantially free of visible particles after storage at 25° C., 30° C. or 40° C. for at least one, two or three months. Visible particles are suitably detected using the 2.9.20. European Pharmacopoeia Monograph (Particulate Contamination: Visible Particles).

Suitably the aqueous solution formulation of the invention is sufficiently stable such that the concentration of related species remains low upon extended storage.

In one embodiment, the aqueous solution formulation of the invention retains at least 94%, e.g. at least 95%, e.g. at least 96%, e.g. at least 97%, e.g. at least 98%, e.g. at least 99% parent Fc fusion protein (by weight of total Fc fusion protein) after storage at 25° C., 30° C. or 40° C. for one, two or three months. The percentage of Fc fusion protein (by weight of total Fc fusion protein) may be determined by size-exclusion chromatography, cation-exchange chromatography, anion-exchange chromatography, reversed-phase chromatography or capillary electrophoresis.

Suitably, the aqueous solution formulation of the invention comprises no more than 6% (by weight of total protein) high molecular weight species after storage at 25° C., 30° C. or 40° C. for at least one, two or three months. In one embodiment, the amount of high molecular weight species increases by no more than 5% (by weight of total Fc fusion protein), preferably no more than 4%, preferably no more than 3%, preferably no more than 2%, preferably no more than 1%, after storage at 25° C., 30° C. or 40° C. for at least one, two or three months. Quantitation of high molecular weight species is as percent by weight of the total Fc fusion protein in the aqueous solution formulation.

Suitably, the aqueous solution formulation of the invention should exhibit an increase in high molecular weight species during storage at 25° C., 30° C. or 40° C. for at least one, two or three months which is at least 10% lower, preferably at least 25% lower, more preferably at least 50% lower, than a corresponding aqueous solution formulation lacking 5-200 mM sulfate ions but otherwise identical, following storage under the same conditions and length of time.

Suitably, the aqueous solution formulation of the invention should exhibit an increase in high molecular weight species during storage at 25° C., 30° C. or 40° C. for at least one, two or three months which is at least 10% lower, preferably at least 25% lower, more preferably at least 50% lower, than a corresponding aqueous solution formulation lacking a 5-200 mM sulfate ions and 1-100 mM multivalent anions other than sulfate but otherwise identical, following storage under the same conditions and length of time.

In one embodiment, the aqueous solution formulation of the invention is a pharmaceutical formulation suitable for administration of a therapeutic Fc fusion protein to a subject in need thereof. Such a formulation can be used in a method for administering the Fc fusion protein to the subject. Suitably the aqueous solution formulation is administered by subcutaneous or intramuscular injection or by intravenous injection or infusion. More suitably the formulation is administered by subcutaneous injection.

When the Fc fusion protein is etanercept, the aqueous solution formulation of the invention is useful for the treatment of autoimmune and inflammatory diseases including arthritis such as rheumatoid arthritis, ankylosing spondylitis, granulomatosis, psoriasis, Crohn's disease, inflammatory bowel disease, atopic dermatitis, asthma and COPD.

In another embodiment, the invention provides a packaged pharmaceutical formulation suitable for administration to a subject in need thereof. The pharmaceutical formulation comprises an aqueous solution formulation of the invention. The pharmaceutical formulation is preferably packaged in a vial suitable for introduction of a needle for removal of the solution. The vial may be single-dose or multi-dose. The pharmaceutical formulation for a multi-dose vial suitably comprises a preservative. In one embodiment, the pharmaceutical formulation is packaged in a glass vial with a rubber stopper. The packaged pharmaceutical formulation can be provided as a kit, further comprising instructions for use and, optionally, a syringe suitable for intramuscular or subcutaneous administration. Alternatively, the packaged pharmaceutical formulation can be provided in the form of a pre-filled single-use disposable syringe suitable for intramuscular or subcutaneous administration. A pre-filled auto-injector device would also be suitable for intramuscular or subcutaneous administration. A further aspect of the invention is a pre-filled single-use syringe for injection comprising the aqueous solution formulation of the invention.

Alternatively, the packaged pharmaceutical formulation can be provided in the form of a pre-filled multi-dose pen (e.g. a disposable pen) suitable for intramuscular or subcutaneous administration. Thus, a further aspect of the invention is a pre-filled multi-dose pen for injection comprising the aqueous solution formulation of the invention. The pharmaceutical formulation for such a multi-dose pen suitably comprises a preservative.

Components of the aqueous solution formulation of the invention are suitably pharmaceutically acceptable. The term “pharmaceutically acceptable”, as used herein, refers to components of a pharmaceutical formulation which are suitable for the intended use and mode of administration to the body of a human or an animal, such as a mammal, without undue adverse consequences, such as toxicity, irritation, and allergic response and with a reasonable risk/benefit ratio.

A further aspect of the invention is a method of stabilizing an Fc fusion protein in an aqueous solution formulation comprising the step of adding to the formulation sulfate ions at a concentration of 5-200 mM thereby to stabilize the fusion protein. A further aspect of the invention is a method of stabilizing an Fc fusion protein in an aqueous solution formulation comprising the step of adding to the formulation sulfate ions at a concentration of 5-200 mM and multivalent anions other than sulfate at a concentration of 1-100 mM thereby to stabilise the fusion protein. The aforementioned methods may, for example, be methods for inhibiting formation of high molecular weight species of the Fc fusion protein during storage or methods for inhibiting formation of low molecular weight species or methods for inhibiting formation of related species of the Fc fusion protein during storage.

The invention also provides use of sulfate ions at a concentration of 5-200 mM for stabilizing an Fc fusion protein to storage in an aqueous solution formulation.

The invention also provides use of a mixture of sulfate ions at a concentration of 5-200 mM and multivalent anions other than sulfate at a concentration of 1-100 mM for stabilizing to storage an Fc fusion protein in an aqueous solution formulation.

Further specific aspects of the invention include:

(A) An aqueous solution formulation comprising (i) an Fc fusion protein, for example etanercept;
(i) sulfate ions at a concentration of 5-200 mM, for example 50-100 mM or 50-90 mM to stabilise the fusion protein;
(ii) multivalent anions other than sulfate at a concentration of 1-100 mM e.g. 10-50 or 10-60 mM, for example multivalent anions selected from citrate, maleate and phosphate ions;
(iii) optionally one or more preservatives; and
(iv) optionally one or more uncharged tonicity modifiers;
which formulation is free of amino acids selected from arginine, lysine and proline, and salts thereof, and which formulation is free of magnesium ions.
(B) An aqueous solution formulation comprising (i) an Fc fusion protein, for example etanercept;
(i) sulfate ions at a concentration of 5-200 mM, for example 50-100 mM or 50-90 mM to stabilise the fusion protein;
(ii) citrate ions at a concentration of 5-60 mM e.g. 10-60 mM;
(iii) phosphate ions at a concentration of 1-10 mM e.g. 1-5 mM e.g. 3-5 mM;
(iv) optionally one or more preservatives; and
(v) optionally one or more uncharged tonicity modifiers;
which formulation is free of amino acids selected from arginine, lysine and proline, and salts thereof, and which formulation is free of magnesium ions.
(C) An aqueous solution formulation comprising (i) an Fc fusion protein, for example etanercept;
(i) sulfate ions at a concentration of 5-200 mM, for example 50-100 mM or 50-90 mM to stabilise the fusion protein;
(ii) maleate ions at a concentration of 5-60 mM e.g. 10-60 mM;
(iii) phosphate ions at a concentration of 1-10 mM e.g. 1-5 mM e.g. 3-5 mM;
(iv) optionally one or more preservatives; and
(v) optionally one or more uncharged tonicity modifiers;
which formulation is free of amino acids selected from arginine, lysine and proline, and salts thereof, and which formulation is free of magnesium ions.
(D) An aqueous solution formulation comprising (i) an Fc fusion protein, for example etanercept;
(i) sulfate ions at a concentration of 5-200 mM, for example 50-100 mM or 50-90 mM to stabilise the fusion protein;
(ii) maleate ions at a concentration of 5-60 mM e.g. 10-30 e.g. 10-20 mM;
(iii) citrate ions at a concentration of 5-60 mM e.g. 10-30 e.g. 10-20 mM;
(iv) optionally one or more preservatives; and
(v) optionally one or more uncharged tonicity modifiers;
which formulation is free of amino acids selected from arginine, lysine and proline, and salts thereof, and which formulation is free of magnesium ions.
(E) An aqueous solution formulation consisting essentially of (i) an Fc fusion protein, for example etanercept;
(i) sulfate ions at a concentration of 5-200 mM, for example 50-100 mM or 50-90 mM to stabilise the fusion protein;
(ii) multivalent anions other than sulfate at a concentration of 1-100 mM e.g. 10-50 or 10-60 mM, for example multivalent anions selected from citrate, maleate and phosphate ions;
(iii) optionally one or more preservatives; and
(iv) optionally one or more uncharged tonicity modifiers; which formulation is free of amino acids selected from arginine, lysine and proline, and salts thereof, and which formulation is free of magnesium ions.
(F) An aqueous solution formulation consisting essentially of (i) an Fc fusion protein, for example etanercept;
(i) sulfate ions at a concentration of 5-200 mM, for example 50-100 mM or 50-90 mM to stabilise the fusion protein;
(ii) citrate ions at a concentration of 5-60 mM e.g. 10-60 mM;
(iii) phosphate ions at a concentration of 1-10 mM e.g. 1-5 mM e.g. 3-5 mM;
(iv) optionally one or more preservatives; and
(v) optionally one or more uncharged tonicity modifiers; which formulation is free of amino acids selected from arginine, lysine and proline, and salts thereof, and which formulation is free of magnesium ions.
(G) An aqueous solution formulation consisting essentially of (i) an Fc fusion protein, for example etanercept;
(i) sulfate ions at a concentration of 5-200 mM, for example 50-100 mM or 50-90 mM to stabilise the fusion protein;
(ii) maleate ions at a concentration of 5-60 mM e.g. 10-60 mM;
(iii) phosphate ions at a concentration of 1-10 mM e.g. 1-5 mM e.g. 3-5 mM;
(iv) optionally one or more preservatives; and
(v) optionally one or more uncharged tonicity modifiers; which formulation is free of amino acids selected from arginine, lysine and proline, and salts thereof, and which formulation is free of magnesium ions.
(H) An aqueous solution formulation consisting essentially of (i) an Fc fusion protein, for example etanercept;
(i) sulfate ions at a concentration of 5-200 mM, for example 50-100 mM or 50-90 mM to stabilise the fusion protein;
(ii) maleate ions at a concentration of 5-60 mM e.g. 10-30 e.g. 10-20 mM;
(iii) citrate ions at a concentration of 5-60 mM e.g. 10-30 e.g. 10-20 mM;
(iv) optionally one or more preservatives; and
(v) optionally one or more uncharged tonicity modifiers;
which formulation is free of amino acids selected from arginine, lysine and proline, and salts thereof, and which formulation is free of magnesium ions.
(I) Any of aqueous solution formulations (A) to (H) wherein an uncharged tonicity modifier is present at a concentration of about 50-500 mM e.g. about 50-300 mM.
(J) An aqueous solution formulation comprising (i) an Fc fusion protein, for example etanercept;
(i) sulfate ions at a concentration of 5-200 mM, for example 50-100 mM or 50-90 mM to stabilise the fusion protein;
(ii) multivalent anions other than sulfate at a concentration of 1-100 mM e.g. 10-50 or 10-60 mM, for example multivalent anions selected from citrate, maleate and phosphate ions;
(iii) optionally one or more preservatives; and
(iv) optionally one or more charged tonicity modifiers;
which formulation is free of amino acids selected from arginine, lysine and proline, and salts thereof, and which formulation is free of magnesium ions.
(K) An aqueous solution formulation comprising (i) an Fc fusion protein, for example etanercept;
(i) sulfate ions at a concentration of 5-200 mM, for example 50-100 mM or 50-90 mM to stabilise the fusion protein;
(ii) citrate ions at a concentration of 5-60 mM e.g. 10-60 mM;
(iii) phosphate ions at a concentration of 1-10 mM e.g. 1-5 mM e.g. 3-5 mM;
(iv) optionally one or more preservatives; and
(v) optionally one or more charged tonicity modifiers;
which formulation is free of amino acids selected from arginine, lysine and proline, and salts thereof, and which formulation is free of magnesium ions.
(L) An aqueous solution formulation comprising (i) an Fc fusion protein, for example etanercept;
(i) sulfate ions at a concentration of 5-200 mM, for example 50-100 mM or 50-90 mM to stabilise the fusion protein;
(ii) maleate ions at a concentration of 5-60 mM e.g. 10-60 mM;
(iii) phosphate ions at a concentration of 1-10 mM e.g. 1-5 mM e.g. 3-5 mM;
(iv) optionally one or more preservatives; and
(v) optionally one or more charged tonicity modifiers;
which formulation is free of amino acids selected from arginine, lysine and proline, and salts thereof, and which formulation is free of magnesium ions.
(M) An aqueous solution formulation consisting essentially of (i) an Fc fusion protein, for example etanercept;
(i) sulfate ions at a concentration of 5-200 mM, for example 50-100 mM or 50-90 mM to stabilise the fusion protein;
(ii) multivalent anions other than sulfate at a concentration of 1-100 mM e.g. 10-50 or 10-60 mM, for example multivalent anions selected from citrate, maleate and phosphate ions;
(iii) optionally one or more preservatives; and
(iv) optionally one or more charged tonicity modifiers; which formulation is free of amino acids selected from arginine, lysine and proline, and salts thereof, and which formulation is free of magnesium ions.
(N) An aqueous solution formulation consisting essentially of (i) an Fc fusion protein, for example etanercept;
(i) sulfate ions at a concentration of 5-200 mM, for example 50-100 mM or 50-90 mM to stabilise the fusion protein;
(ii) citrate ions at a concentration of 5-60 mM e.g. 10-60 mM;
(iii) phosphate ions at a concentration of 1-10 mM e.g. 1-5 mM e.g. 3-5 mM;
(iv) optionally one or more preservatives; and
(v) optionally one or more charged tonicity modifiers; which formulation is free of amino acids selected from arginine, lysine and proline, and salts thereof, and which formulation is free of magnesium ions.
(O) An aqueous solution formulation consisting essentially of (i) an Fc fusion protein, for example etanercept;
(i) sulfate ions at a concentration of 5-200 mM, for example 50-100 mM or 50-90 mM to stabilise the fusion protein;
(ii) maleate ions at a concentration of 5-60 mM e.g. 10-60 mM;
(iii) phosphate ions at a concentration of 1-10 mM e.g. 1-5 mM e.g. 3-5 mM;
(iv) optionally one or more preservatives; and
(v) optionally one or more charged tonicity modifiers; which formulation is free of amino acids selected from arginine, lysine and proline, and salts thereof, and which formulation is free of magnesium ions.
(P) An aqueous solution formulation comprising (i) an Fc fusion protein, for example etanercept;
(i) sulfate ions at a concentration of 5-200 mM, for example 50-100 mM or 50-90 mM to stabilise the fusion protein;
(ii) maleate ions at a concentration of 5-60 mM e.g. 10-30 e.g. 10-20 mM;
(iii) citrate ions at a concentration of 5-60 mM e.g. 10-30 e.g. 10-20 mM;
(iv) optionally one or more preservatives; and
(v) optionally one or more charged tonicity modifiers;
which formulation is free of amino acids selected from arginine, lysine and proline, and salts thereof, and which formulation is free of magnesium ions.
(Q) An aqueous solution formulation consisting essentially of (i) an Fc fusion protein, for example etanercept;
(i) sulfate ions at a concentration of 5-200 mM, for example 50-100 mM or 50-90 mM to stabilise the fusion protein;
(ii) maleate ions at a concentration of 5-60 mM e.g. 10-30 e.g. 10-20 mM;
(iii) citrate ions at a concentration of 5-60 mM e.g. 10-30 e.g. 10-20 mM;
(iv) optionally one or more preservatives; and
(v) optionally one or more charged tonicity modifiers;
which formulation is free of amino acids selected from arginine, lysine and proline, and salts thereof, and which formulation is free of magnesium ions.
(R) Any of aqueous solution formulations (J) to (Q) wherein a charged tonicity modifier such as sodium chloride is present at a concentration of about 50-250 mM.
(S) Any of aqueous solution formulations (A) to (R) at a pH of about 5.0 to about 6.0 e.g. about 5.0 to about 5.7 e.g. about 5.3.
(T) Any of aqueous solution formulations (A) to (R) at a pH of about 6.0 to about 7.0 e.g. about 6.0 to about 6.5 e.g. about 6.3.

### Abbreviations

PEG polyethylene glycol
HMWS high molecular weight species
SEC size exclusion chromatography
CEX cation-exchange chromatography

### EXAMPLES

### Materials

EDTA disodium salt (Mw 372 Da), 1,2-propanediol (Mw 76 Da), glycerol (Mw 92 Da), mannitol (Mw 182 Da), NaCl (Mw 58 Da), trehalose (Mw 342 Da) were obtained from Sigma Aldrich.

### Methods of Assessing Stability of an Fc Fusion Protein

### (a) Visual Assessment

Visible particles are suitably detected using the 2.9.20. European Pharmacepoeia Monograph (Particulate Contamination: Visible Particles). The apparatus required consists of a viewing station comprising:
a matt black panel of appropriate size held in a vertical position
a non-glare white panel of appropriate size held in a vertical position next to the black panel
an adjustable lampholder fitted with a suitable, shaded, white-light source and with a suitable light diffuser (a viewing illuminator containing two 13 W fluorescent tubes, each 525 mm in length, is suitable). The intensity of illumination at the viewing point is maintained between 2000 lux and 3750 lux.

Any adherent labels are removed from the container and the outside washed and dried. The container is gently swirled or inverted, ensuring that air bubbles are not introduced, and observed for about 5 s in front of the white panel. The procedure is repeated in front of the black panel. The presence of any particles is recorded.

The visual scores are ranked as follows:
Visual score 1: Clear solution, virtually free of particles
Visual score 2: ˜5 very small particles
Visual score 3: ˜10-20 very small particles
Visual score 4: 20-50 particles, including larger particles
Visual score 5: >50 particles, including larger particles

Whilst the particles in samples with visual scores 4 and 5 are clearly detectable on casual visual assessment under normal light, samples with visual score 1-3 generally appear as clear solutions on the same assessment. Samples with visual scores 1-3 are considered to be “Pass”; samples with visual score 4-5 are considered to be “Fail”.

### (b) Size Exclusion Chromatography (SEC)

The amount of high molecular weight species (HMWS) and low molecular weight species (LMWS) is measured using a 300×7.8 mm 53000 (or equivalent) size-exclusion column with a guard column. The mobile phase is potassium phosphate pH 6.5, with a flow rate of 0.4 ml/min, injection volume of 1 μl and detected at 210 and 280 nm. The results are expressed as % main peak (i.e. native protein), % HMWS and % LMWS.

### (c) Cation-Exchange Chromatography (CEX)

The amount of related species is measured using a Protein-Pak Hi Res SP column. Mobile phase A is 20 mM sodium phosphate (pH 6.5); mobile phase B is 20 mM sodium phosphate+0.5 M NaCl (pH 6.0). The following gradient elution is used: 0 min-100% A, 4 min-80% A, 10 min-55% A, 12 min-0% A. Flow rate of 1.0 ml/min; injection volume is 3 μl, with UV detection at 214 nm. The results are expressed as % main peak (i.e. native protein), % acidic species and % basic species. % Related species=% acidic species+% basic species.

### Example 1

### Stability of Etanercept Formulations

The stability of etanercept (50 mg/mL) was tested in the formulations shown in the table below (formulations F1-1-F1-6) using the SEC method. The pH of all formulations tested was adjusted to 6.3. Formulations F1-1-F1-3 were prepared as approximately isotonic so the concentrations of the key excipients tested vary depending on their relative contribution to osmolarity. Formulations F1-4-F1-6 combine excipients and are hypertonic.

| | Sodium | Sodium | | Sodium | Sodium |
|---|---|---|---|---|---|
| | phosphate | chloride | Sucrose | sulphate | citrate |
| Formulation | (mM) | (mM) | (mM) | (mM) | (mM) |
| F1-1 | 5 | 150 | | | |
| F1-2 | 5 | | 300 | | |
| F1-3 | 5 | | | 100 | |
| F1-4 | 5 | 150 | | 100 | |
| F1-5 | 5 | | 300 | 100 | |
| F1-6 | 5 | | | 100 | 50 |

The stability of formulations F1-1-F1-6 is shown in the table below. The table shows the retention of the main peak on the SEC chromatogram following storage at 25° C. and 40° C.

| | | | | | Δ | Δ |
|---|---|---|---|---|---|---|
| Formu- | | 4 weeks | 4 weeks | 12 weeks | (0-4 weeks | (0-12 weeks |
| lation | 0 weeks | (40° C.) | (25° C.) | (25° C.) | at 40° C.) | at 25° C.) |
| F1-1 | 98.29% | 90.02% | 97.23% | 95.63% | 8.27% | 2.66% |
| F1-2 | 98.39% | 90.99% | 97.28% | 96.02% | 7.40% | 2.37% |
| F1-3 | 98.41% | 93.82% | 97.83% | 96.38% | 4.59% | 2.03% |
| F1-4 | 98.27% | 93.84% | 97.81% | 96.39% | 4.43% | 1.88% |
| F1-5 | 98.29% | 93.72% | 97.90% | 96.29% | 4.57% | 2.00% |
| F1-6 | 98.40% | 94.49% | 97.97% | 96.79% | 3.91% | 1.61% |

The results show that the use of sucrose as a tonicity modifier resulted in a slightly better stability compared with the use of sodium chloride (F1-1 vs. F1-2). However, using sodium sulfate resulted in notably better stability compared with sodium chloride or sucrose.

Combining sodium sulfate with either sodium chloride or sucrose led to only a modest improvement in stability over the use of sodium sulfate alone (F1-4 vs. F1-3). However, it was found that combining sodium sulfate with sodium citrate resulted in further more significant stability improvement (F1-6 vs. F1-3).

### Example 2

### Stability of Etanercept Formulations

The effect multivalent anions on the stability of etanercept (50 mg/mL) in the presence of sodium sulfate was further investigated in the formulations shown in the table below (formulations F2-1-F2-9) using the SEC method. The pH of all formulations tested was adjusted to 6.3.

| | Sodium | Sodium | Sodium | Sodium |
|---|---|---|---|---|
| | phosphate | maleate | citrate | sulphate |
| Formulation | (mM) | (mM) | (mM) | (mM) |
| F2-1 | 5 | | | 50 |
| F2-2 | 5 | | | 100 |
| F2-3 | 5 | | 50 | 50 |
| F2-4 | 5 | | 50 | 100 |
| F2-5 | 5 | | 10 | 100 |
| F2-6 | 5 | 50 | | 100 |
| F2-7 | 5 | 10 | | 100 |
| F2-8 | 5 | 50 | 50 | 100 |
| F2-9 | 5 | 10 | 10 | 100 |

The stability of formulations F2-1-F2-9 is shown in the table below. The table shows the retention of the main peak on the SEC chromatogram following storage at 25° C. and 40° C.

| | | | | Δ | Δ |
|---|---|---|---|---|---|
| | | 4 weeks | 12 weeks | (0-4 weeks | (0-12 weeks |
| Formulation | 0 weeks | (40° C.) | (25° C.) | at 40° C.) | at 25° C.) |
| F2-1 | 98.11% | 92.16% | 95.74% | 5.95% | 2.37% |
| F2-2 | 98.27% | 93.49% | 96.09% | 4.78% | 2.18% |
| F2-3 | 98.34% | 93.00% | 95.94% | 5.34% | 2.40% |
| F2-4 | 98.27% | 94.16% | 96.50% | 4.11% | 1.77% |
| F2-5 | 98.16% | 93.86% | 96.16% | 4.30% | 2.00% |
| F2-6 | 98.27% | 94.01% | 96.42% | 4.26% | 1.85% |
| F2-7 | 98.13% | 93.44% | 96.03% | 4.69% | 2.10% |
| F2-8 | 98.35% | 94.99% | 96.84% | 3.36% | 1.51% |
| F2-9 | 98.20% | 94.02% | 96.30% | 4.18% | 1.90% |

The results show that lower concentration of sulfate (50 mM) resulted in a smaller stabilising effect than the use of a higher concentration of sulfate (100 mM) (F2-1 vs. F2-2).

In both cases the stability was further improved by addition of sodium citrate (F2-4 vs. F2-2 and F2-3 vs. F2-1). The effect of sodium citrate was also found to be concentration-dependent in the presence of 100 mM sodium sulfate (F2-5 vs. F2-4)

Addition of sodium maleate (50 mM or 10 mM) to sodium sulfate (100 mM) also resulted in further improvement of stability in a concentration-dependent manner (F2-6 and F2-7 vs. F2-2).

Similarly, addition of a mixture of sodium citrate and sodium maleate resulted in further improvement of stability in a concentration-dependent manner (F2-8 vs. F2-6 and F2-9 vs. F2-7).

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention.

All publications, patents, patent applications, internet sites, and accession numbers/database sequences (including both polynucleotide and polypeptide sequences) cited are herein incorporated by reference in their entirety for all purposes to the same extent as if each individual publication, patent, patent application, internet site, or accession number/database sequence were specifically and individually indicated to be so incorporated by reference.

1. An aqueous solution formulation comprising:
(i) an Fc fusion protein; and
(ii) sulfate ions at a concentration of 5-200 mM to stabilise the Fc fusion protein;
which formulation is free of amino acids selected from arginine, lysine and proline, and salts thereof, and which formulation is free of magnesium ions.

2. The aqueous solution formulation according to claim 1, wherein the Fc fusion protein is a fusion protein which comprises a heterologous polypeptide which is ligand binding and the Fc portion of an immunoglobulin, particularly IgG1.

3. The aqueous solution formulation according to claim 1, wherein the Fc fusion protein is etanercept, abatacept or belatacept.

4. The aqueous solution formulation according to claim 3, wherein the Fc fusion protein is etanercept.

5. The aqueous solution formulation according to claim 1, wherein the sulfate ions are present in the formulation at a concentration of 25-150 mM e.g. 25-100 mM, more suitably 50-100 mM; and/or
wherein the source of the sulfate ions is a sulfate salt e.g. selected from sodium sulfate, potassium sulfate and ammonium sulfate.

6. (canceled)

7. The aqueous solution formulation according to claim 1, wherein the fusion protein is present in the formulation at a concentration of 1-400 mg/ml, suitably 20-100 mg/ml, more suitably about 50 mg/ml.

8. The aqueous solution formulation according to claim 1, further comprising a multivalent anion other than sulfate as a further stabilizing component.

9. The aqueous solution formulation according to claim 8, wherein the multivalent anion is an organic multivalent anion.

10. The aqueous solution formulation according to claim 9, wherein the organic multivalent anion is citrate.

11. The aqueous solution formulation according to claim 9, wherein the organic multivalent anion is selected from succinate, malate and maleate, particularly maleate.

12. The aqueous solution formulation according to claim 9, comprising citrate and maleate as organic multivalent anions.

13. The aqueous solution formulation according to claim 8, wherein the multivalent anion is an inorganic multivalent anion such as phosphate.

14. The aqueous solution formulation according to claim 8, wherein the concentration of the multivalent anion other than sulfate is 1-100 mM, suitably 1-60 mM or 1-50 mM, more suitably 5-60 or 5-50 mM, more suitably 10-60 or 10-50 mM e.g. 30-50 mM.

15. The aqueous solution formulation according to claim 1, wherein the osmolarity of the formulation is 200-500 mOsm/1 e.g. about 300 mOsm/1; and/or wherein the formulation is isotonic.

16. (canceled)

17. The aqueous solution formulation according to claim 1, further comprising an uncharged tonicity modifier.

18. The aqueous solution formulation according to claim 17, wherein the uncharged tonicity modifier is selected from sucrose, trehalose, mannitol, raffinose, lactose, dextrose, sorbitol, lactitol, glycerol, propylene glycol and polyethylene glycol; and/or wherein the uncharged tonicity modifier is present in the formulation at a concentration of 10-1000 mM, suitably 50-300 mM.

19. (canceled)

20. The aqueous solution formulation according to claim 1, further comprising a charged tonicity modifier.

21. The aqueous solution formulation according to claim 20, wherein the charged tonicity modifier is sodium chloride; and/or
wherein the charged tonicity modifier is present in the formulation at a concentration of 25-500 mM, suitably 50-250 mM.

22. (canceled)

23. The aqueous solution formulation according to claim 1, wherein the pH of the formulation is between about pH 4.0 and about pH 7.0, e.g. between about pH 6.0 and about pH 7.0 e.g. between about pH 6.0 and pH 6.5 e.g. about pH 6.3.

24. (canceled)

25. The aqueous solution formulation according to claim 1, further comprising a buffer, wherein the buffer is present at a concentration of about 0.5 mM to about 50 mM, such as about 1 mM to about 20 mM, such as about 1 mM to about 10 mM, e.g. 1 mM to about 5 mM e.g. about 2 mM to about 5 mM.

26. (canceled)

27. The aqueous solution formulation according to claim 1, further comprising a non-ionic surfactant, wherein the non-ionic surfactant is suitably present at a concentration of about 10 μg/mL to about 2000 μg/mL, such as about 50 μg/mL to about 1000 μg/mL, e.g. about 100 μg/mL to about 500 μg/mL.

28. (canceled)

29. The aqueous solution formulation according to claim 1, further comprising a preservative selected from the group consisting of phenol, m-cresol, chlorocresol, benzyl alcohol, propylparaben, methylparaben, benzalkonium chloride and benzethonium chloride, wherein the preservative is suitably present at a concentration of about 0.01 mM to about 100 mM.

30.-31. (canceled)

32. The aqueous solution formulation according to claim 1, which is free of all amino acids, particularly is free of any of the 20 natural amino acids in their L or D isomeric forms.

33. An aqueous solution formulation comprising an Fc fusion protein, for example etanercept;
(i) sulfate ions at a concentration of 5-200 mM, for example 50-100 mM or 50-90 mM to stabilise the fusion protein;
(ii) maleate ions at a concentration of 5-60 mM e.g. 10-30 e.g. 10-20 mM;
(iii) citrate ions at a concentration of 5-60 mM e.g. 10-30 e.g. 10-20 mM;
(iv) optionally one or more preservatives; and
(v) optionally one or more tonicity modifiers;
which formulation is free of amino acids selected from arginine, lysine and proline, and salts thereof, and which formulation is free of magnesium ions;
wherein suitably the pH of the formulation is between about pH 6.0 and about pH 7.0 e.g. between about pH 6.0 and pH 6.5 e.g. about pH 6.3.

34. (canceled)

35. The aqueous solution formulation according to claim 1, which is a pharmaceutical formulation.

36. (canceled)

37. The aqueous solution formulation according to claim 1, wherein the formulation is for administration by subcutaneous or intramuscular injection or by intravenous injection or infusion.

38. A pre-filled syringe or a pre-filled multi-dose pen for injection comprising the aqueous solution formulation according to claim 1.

39. (canceled)

40. A method of stabilizing an Fc fusion protein in an aqueous solution comprising the step of adding to the formulation sulfate ions at a concentration of 5-200 mM thereby to stabilise the fusion protein.

41. The method of claim 40, wherein the method is a method for inhibiting formation of high molecular weight species of the Fc fusion protein during storage; or
wherein the method is a method for inhibiting formation of low molecular weight species of the Fc fusion protein during storage; or
wherein the method is a method for inhibiting formation of related species of the Fc fusion protein during storage.

42.-44. (canceled)
